(19) United States
(12) Patent Application Publication
(10) Pub. No.: US 2025/0186388 A1
(43) Pub. Date: Jun. 12, 2025

(54) ACTIVE SUBSTANCES FOR MEDICAL USE

(71) Applicant: INFLAMED PHARMA GMBH, Jena (DE)

(72) Inventors: Susanne Vogelsang, Jena (DE); Beatrice Engert, Jena (DE)

(21) Appl. No.: 17/995,327

(22) PCT Filed: Apr. 2, 2021
(86) PCT No.: PCT/EP2021/058787

§ 371 Date: Oct. 3, 2022

(30) Foreign Application Priority Data
Apr. 2, 2020 (LU) LU101724
Feb. 18, 2021 (EP) 21157974.3

(51) Int. Cl. A61K 31/245

(52) U.S. Cl. CPC A61K 31/245

(57) ABSTRACT

The subject matter of the present invention relates to an amine (AM) according to the general formula (I); a carbonic acid adduct (KA) and a pharmaceutical composition (PZ) for use in the treatment of atypical pneumonia and for use in the therapy of viral diseases.

### TECHNICAL FIELD

The subject matter of the present invention relates to an amine (AM) according to the general formula (I); a carbonic acid adduct (KA) and a pharmaceutical composition (PZ) for use in the treatment of atypical pneumonia and in the therapy of viral diseases.

### TECHNICAL BACKGROUND

Procaine and structurally related compounds according to the general formula (I) are used as local anaesthetics. Oral application for systematic pain reduction is not possible because procaine hydrochloride, due to its ionic structure, cannot pass through the intestinal wall. Procaine hydrochloride is appreciably only used parenterally. The procaine is practically not changed in the stomach when administered orally. The higher the pH, the higher the adsorption rate of procaine in the intestine.

Carbonic acid adducts of procaine and structurally related local anaesthetics are outwardly neutral, therefore membrane-permeable and can also pass through the intestinal wall. Due to the better penetration into the tissue, procaine in the form of carbonic acid adducts evades rapid degradation by pseudocholinesterase in the plasma. Therefore, carbonic acid adducts of procaine and structurally related local anaesthetics can be used in a more versatile manner than procaine hydrochloride.

WO2006/007835 A2 discloses ammonium salts and stable storable ammonium salt mineral clathrates with acidic dibasic acid residues such as hydrogen carbonate, processes for their preparation, and pharmaco-medical and chemically synthetic applications for these compounds. The use of procaine and structurally related local anaesthetics or carbonic acid adducts thereof in the treatment of atypical pneumonia is not disclosed.

The aim of application DE 10 2013 015 035 A1, which refers directly to WO2006/007835 A2, is to improve some of the disadvantages of the procaine carbonic acid mineral salt clusters described above by developing suitable formulations in such a way that they become suitable for use as medicinal products and thus meet higher requirements with regard to stability, effect and acceptance. For this purpose, a process for the preparation of carbonic acid mineral salt clusters of procaine is described, as well as its use in formulations for parenteral application, inhalation solutions, ointments and tablets. Analogously to WO2006/007835 A2, the document discloses that both salt and CO₂ are present in the reaction solution for the preparation of the procaine carbonic acid mineral salt clusters. DE 10 2013 015 035 A1 also fails to disclose the use of procaine and structurally related local anaesthetics or carbonic acid adducts thereof in the treatment of atypical pneumonia.

WO2019048590 likewise discloses carbonic acid adducts based on amines, in particular also on procaine and structurally related local anaesthetics, as well as pharmaceutical preparations thereof and processes for their preparation. In addition, medical uses of the carbon adducts are disclosed, but not the use in the treatment of atypical pneumonia.

Atypical pneumonia refers to an inflammation of the alveolar space and/or the interstitial lung tissue, which can be triggered by viruses, bacteria or also fungi, among other things. During inflammation [1], adrenaline is released, which leads to a constriction of the arterioles and dilation of venules. Then, exudation takes place. The walls of the capillaries become more permeable for the passage of protein and neutrophils. Inflammatory swelling develops. The permeability of the vessel walls is increased by vascular mediators, such as histamine, prostaglandins, kinins and serotonin, so that blood congestion occurs. The nerve endings are irritated by the increased tissue pressure, peptides and lactic acid, resulting in pain. Now the cellular reaction begins, i.e. neutrophils emerge from the vessels and reach the site of the stimulus or damage mainly through chemotaxis (acidic environment). Phagocytosis occurs. The resulting decay products can trigger fever and cause pus. Furthermore, more mast cells flood into the inflamed tissue.

A possible fatal complication, especially in conjunction with atypical pneumonia, is an excessive immune response that can lead to the development of acute respiratory distress syndrome (ARDS) [2]. The increase in the permeability of the vessels and the lung damage in the course of the inflammation lead to the formation of interstitial pulmonary oedema. Inflammatory factors such as TNF-α, IL-6 and IL-8 trigger the migration of neutrophil granulocytes. These release free radicals such as O, H₂O₂, OH and HOCl and lytic enzymes which further intensify the inflammatory response. Under the influence of the inflammatory mediators, a pronounced “capillary leak” occurs, leading to the formation of alveolar oedema. This destroys the surfactant on the alveolar surfaces, resulting in microatelectasis. The gas exchange between the lungs and the blood is disturbed.

S. Pecher et al [3] discloses that local anaesthetics such as lidocaine have an anti-inflammatory effect. Pecher et al. cite studies by Mikawa et al. on rabbits incubated with E. coli and administered lidocaine. It was found that lidocaine administration led to an increase in pₐO₂ and improved lung mechanics in the sense of improved compliance and reduced resistance. In addition, there was a reduced incidence of pulmonary oedema compared to a comparison group. In the bronchoalveolar lavage, there were fewer leucocytes and a lower albumin content in the lidocaine group. The formation of haemorrhages, alveolar septal thickening and the number of inflammatory cells in the alveolar space could be visibly reduced by lidocaine. However, S. Pecher does not disclose the use of procaine or structurally related local anaesthetics or carbonic acid adducts thereof in the treatment of atypical pneumonia, especially atypical pneumonia caused by viruses or fungi.

Worldwide, research activities are running at full speed to find medicaments for the treatment of COVID-19 diseases. It is already becoming apparent that there will not be “one medicament”, as the courses are very different and the phase of the disease also plays an essential role in the choice of medicament treatment. In the early phase, the focus is on preventing invasion and reducing viral replication. In the second and third phase, the reduction of inflammation and thus the prevention of the cytokine storm is of greatest importance. Currently, the generalised inflammatory process (especially in the vessels) is becoming more and more central to the pathophysiology of a Covid-19 infection. However, it also opens up therapeutic options, in addition to the use of vaccines as a preventive measure and specific antiviral treatment strategies, which are rare. In addition, substances directed against the viruses themselves usually cause rapid development of resistance, so that the substances become ineffective. Therefore, new antiviral strategies target virus-supporting cellular factors that are used by the viruses to support their own replication and spread. Further points of attack are the inhibition of excessive immune reactions or the active resolution of inflammatory processes.

In view of the recent pandemic triggered by the Sars-CoV-2 virus and the sometimes fatal course, including the development of acute respiratory distress syndrome (ARDS), it is clear that there is a further need for therapeutic agents that help to bring about at least a milder course of atypical pneumonia, especially atypical pneumonia triggered by viruses and fungi. There is also a need for further antiviral substances, especially for the treatment of COVID-19 diseases.

### SUMMARY OF THE INVENTION

The invention relates to an amine (AM) according to the general formula (I)

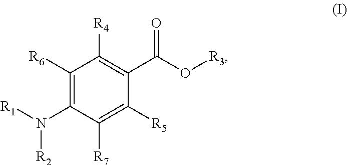

wherein in formula (I)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O—(C₁₋₁₀)alkyl;
wherein the amine according to formula (I) can optionally also be used in the form of a salt, for use in the treatment of atypical pneumonia and in the therapy of viral diseases.

In a further aspect, the invention relates to a carbonic acid adduct (KA) comprising at least one structural element according to the general formula (II), (III) and/or (IV)

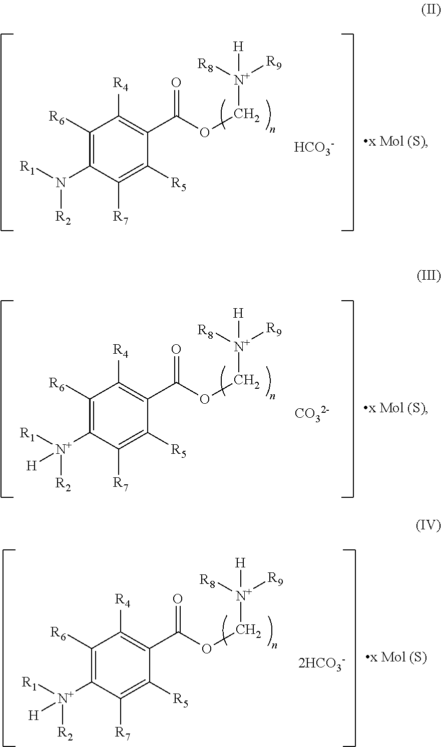

wherein in formulas (II), (III), and (IV)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl; x is 0.5 to 30;
(S) is a salt,
for use in the treatment of atypical pneumonia and in the therapy of viral diseases.

In another aspect, the invention relates to a carbonic acid adduct (KA) comprising carbonic acid, at least one amine (AM) according to the general formula (I) and at least one salt (S),

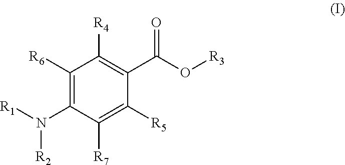

wherein in formula (I)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2;
R₈ is (C₁₋₁₀)alkyl, preferably (C₁₋₂)alkyl;
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
wherein the at least one amine according to formula (I) can optionally also be used in the form of a salt;
preparable by a process comprising the steps:
a) providing a solution (A) comprising at least one solvent and CO₂ dissolved in the at least one solvent,
optionally b) dissolving a base (BA), other than the amine (AM), in the solution (A) to obtain the solution (A1),
c) dissolving the at least one amine (AM) in the solution (A) or (A1) to obtain the solution (B),
d) freezing the solution obtained after completion of step c),
(e) storing the solution frozen in step d) at −100 to 0° C. for not more than 4 days;
for use in the treatment of atypical pneumonia and in the therapy of viral diseases.

The invention further relates to a pharmaceutical composition comprising the amine, or the carbonic acid adduct, for use in the treatment of atypical pneumonia and in the therapy of viral diseases.

### FIGURE DESCRIPTION

FIG. 1: A: Test for antiviral activity against influenza viruses. B and C: Test for antiviral activity against SARS-CoV-2. ProcCluster corresponds to carbonic acid adduct (KA) prepared according to Example 1.

### DETAILED DESCRIPTION OF THE INVENTION

The invention is directed to an amine (AM) according to the general formula (I).

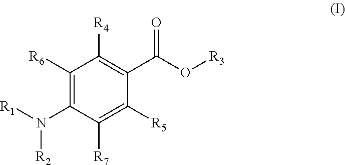

wherein in formula (I)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O—(C₁₋₁₀)alkyl; wherein the amine according to formula (I) can optionally also be used in the form of a salt, for use in the treatment of atypical pneumonia and in the therapy of viral diseases.

In one embodiment in formula (I) R₁, R₂, R₃, R₄, R₅, R₆ is H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;

In the present invention, definitions such as (C₁₋₁₀)alkyl, as defined for example for the group R₁ of formula (I), mean that this substituent (group) is a saturated alkyl group with a carbon number of 1 to 10. The alkyl group can be both linear and branched and optionally cyclic. Alkyl groups that have both a cyclic and a linear component also fall within this definition. The same applies to other alkyl groups such as a C₁₋₂ alkyl group. Where appropriate, alkyl groups may also be mono- or polysubstituted with functional groups such as amino, hydroxy, halogen, aryl or heteroaryl. Unless otherwise stated, alkyl groups preferably do not have functional groups as substituents. Examples of alkyl groups are methyl, ethyl, n-propyl, n-butyl, n-pentyl, n-hexyl, n-heptyl, n-octyl, n-nonyl, iso-propyl (also called 2-propyl or 1-methylethyl), iso-butyl, tert-butyl, sec-pentyl, neo-pentyl, 1,2-dimethylpropyl, iso-amyl, iso-hexyl, iso-heptyl.

In the present invention, definitions such as C₂₋₁₀ alkenyl, as defined for example for the group R₁ of formula (I), mean that this substituent (group) is an alkenyl group with a carbon number of 2 to 10 which has at least one unsaturated carbon-carbon bond. The alkenyl group can be linear or branched and optionally cyclic. Alkenyl groups having both a cyclic and a linear component also fall within this definition. The same applies to other alkenyl groups such as a C₂₋₄ alkenyl group. Where appropriate, alkenyl groups can also be mono- or polysubstituted with functional groups such as amino, hydroxy, halogen, aryl or heteroaryl. Preferably, alkenyl groups do not have any other functional groups as substituents. Examples of alkenyl groups are vinyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 3-heptenyl, 4-heptenyl, 1-octenyl, 3-octenyl, 5-octenyl, 1-nonenyl, 2-nonenyl.

In the present invention, the definition aryl means an aromatic or heteroaromatic group. An aromatic group is an aromatic cyclic hydrocarbon which can comprise a ring or a ring system of multiple fused rings. For example, the aromatic group may be monocyclic, bicyclic or tricyclic. Preferably, the monocyclic aromatic group forms a 5- or 6-membered ring. Preferably, the bicyclic aromatic ring forms a 9- or 10-membered ring. Preferably, the tricyclic aromatic ring forms a 13- or 14-membered ring. A definition such as (C₅-C₁₄)aryl means that the aryl group comprises 5 to 14 carbon atoms. Preferably, the aryl group contains 3 to 14, more preferably 4 to 6 carbon atoms. Where appropriate, aryl groups can also be mono- or polysubstituted with functional groups such as alkyl, alkenyl, amino, cyano, —CF₃, hydroxy, halogen, aryl or heteroaryl, preferably the aryl groups have no further substituents. Examples of aromatic groups are phenyl and naphthyl.

In the present invention, the definition heteroaryl means that it is a heteroaromatic group. Heteroaromatic ring means that in an aromatic group as defined above, the ring system of which is formed by carbon atoms, one or more of these carbon atoms are replaced by heteroatoms such as O, N or S. A definition such as (C₅-C₁₀)heteroaryl is based on the corresponding definition for the aryl group, and means that the heteroaryl group has 5 to 10 atoms in the ring. However, as defined above, 1 or more carbon atoms are replaced by heteroatoms. This means that the (C₅-C₁₀)heteroaryl group has 5 to 10 atoms in the ring, but not all of them are carbon atoms. Therefore, furanyl, for example, would be a C₅-heteroaryl group. Where appropriate, heteroaryl groups can also be mono- or polysubstituted with functional groups such as alkyl, alkenyl, amino, cyano, —CF₃, hydroxy, halogen, aryl or heteroaryl, preferably the heteroaryl groups have no other substituents. Examples of heteroaromatic groups that fall within the definition of aryl in the present invention are furanyl, thienyl, oxazolyl, pyrazolyl, pyridyl and indolyl.

In the present invention, the definition halogen, as defined for example above for the group R₄ for formula (I), means that it is a chlorine, bromine, iodine or fluorine substituent. Preferably, it is a chlorine or fluorine substituent.

Preferably, the amine (AM) according to the general formula (I) is selected from the group consisting of 4-aminobenzoic acid-2-(N,N-diethylamino)ethyl ester (procaine), 4-aminobenzoic acid ethyl ester (benzocaine), 2-(diethylamino)ethyl-4-amino-2-chlorobenzoate (chloroprocaine), 4-amino-3-butoxybenzoic acid-2-diethylaminoethyl ester (oxybuprocaine), (2-(dimethylamino)ethyl)-4-(butylamino)benzoate (tetracaine), more preferably 4-aminobenzoic acid-2-(N,N-diethylamino)ethyl ester (procaine).

The amines (AM) according to the general formula (I) and those specifically described above are in medical use as local anaesthetics and are commercially available accordingly.

As already stated above, atypical pneumonia, particularly within the present invention, refers to pneumonia caused by viruses, bacteria or fungi, preferably by viruses. As also explained above, atypical pneumonia may be associated with the development of acute respiratory distress syndrome (ARDS) as a result of hyperinflammation in the lungs in the context of atypical pneumonia.

In one embodiment, the atypical pneumonia is caused by bacteria. Preferably, the bacteria are selected from the group consisting of Mycoplasma, Legionella, and Chlamydia.

In another embodiment, atypical pneumonia is caused by fungi. Preferably, the fungi are selected from a group consisting of Aspergillus, Pneumocystis and Candida.

In another embodiment, the atypical pneumonia is caused by viruses. The pneumonia is preferably caused by a virus selected from coronaviruses, influenza viruses, adenoviruses and respiratory syncytial virus. More preferred is the virus SARS CoV-2.

Use in the therapy of viral diseases involves the exploitation of an antiviral effect. Typically, antiviral active substances inhibit the developmental and replication cycle instead of attacking the viruses directly.

Without committing to a specific theoretical explanation, it is currently believed that the observed antiviral effect of the compounds of the invention is probably mediated preferably indirectly by interaction with metabolic pathways of the infected organism that are required for the replication of the virus.

The therapy of viral diseases preferably concerns diseases caused by RNA viruses, more preferably coronaviruses, influenza viruses, adenoviruses and respiratory syncytial virus, even more preferably influenza viruses and coronaviruses, particularly preferably coronaviruses, very particularly preferably SARS CoV-2.

In one embodiment, patients who do not require intensive medical treatment are treated in the therapy of viral diseases.

In one embodiment, the amine (AM), carbonic acid adduct (KA) or pharmaceutical composition (PZ), especially when used in the therapy of viral diseases, is not administered in combination with dexamethasone.

In a further embodiment, the pharmaceutical composition (PZ) does not comprise dexamethasone, in particular in the use in the therapy of viral diseases.

Furthermore, the invention relates to a carbonic acid adduct (KA) comprising at least one structural element according to the general formula (II), (III) and/or (IV)

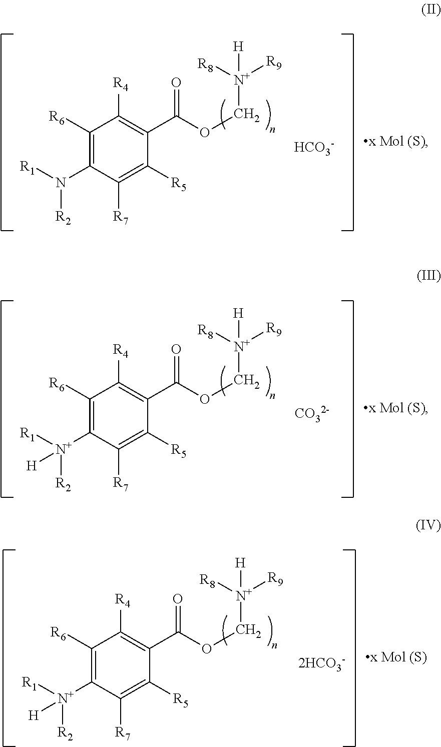

wherein in formulas (II), (III), and (IV)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
x is 0.5 to 30;
(S) is a salt;
for use in the treatment of atypical pneumonia and in the therapy of viral diseases as defined above.

In one embodiment in formula (I) R₁, R₂, R₃, R₄, R₅, R₆ is H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;

The salt (S) comprises at least one cation selected from Na⁺, K⁺, Li⁺, Mg²⁺, Zn²⁺, Fe²⁺, Fe³⁺ and Mn²⁺, preferably Na⁺. Furthermore, the salt (S) comprises at least one anion selected from Cl⁻, Br⁻, J⁻, F⁻, SO₄²⁻, SO₃²⁻, HSO₄, HSO₃_{^{−}}, HCO₃_{^{−}}, CO₃²⁻, PO₄³⁻, HPO₄²⁻, H₂PO₄, SiO₄⁴⁻, AlO₂_{^{−}}, SiO₃_{^{−}} and/or [AlO₂)₁₂(SiO₂)₂]²⁻, preferably Cl⁻ and Br⁻, more preferably Cl⁻.

The preparation of the carbonic acid adduct (KA) is also disclosed in WO2006/007835, DE 10 2013 015 035 A1 and WO2019/048590, to which reference is hereby made.

Furthermore, the invention relates to a carbonic acid adduct (KA) comprising carbonic acid, at least one amine (AM) according to the general formula (I) and at least one salt (S),

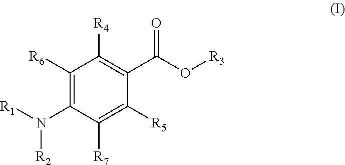

wherein in formula (I)
R₁ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₂ is H, (C₁₋₁₀)alkyl, (C₂₋₁₀)alkenyl, (C₅-C₁₄)aryl or (C₅-C₁₀)heteroaryl, preferably H or (C₁₋₁₀)alkyl, more preferably H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;
R₄ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or halogen;
R₅ is H, halogen, (C₁₋₁₀)alkyl, (C₂₋₁₀)alkenyl, (C₅-C₁₄)aryl, (C₅-C₁₀)heteroaryl or —O—(C₁₋₁₀)alkyl, preferably H, halogen, (C₁₋₁₀)alkyl or —O(C₁₋₁₀)alkyl, more preferably H or halogen;
R₆ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl or —O(C₁₋₁₀)alkyl, preferably H, halogen, (C₁₋₁₀)alkyl or —O(C₁₋₁₀)alkyl, more preferably H or —O(C₁₋₁₀)alkyl;
wherein the at least one amine according to formula (I) can optionally also be used in the form of a salt;
for use in the treatment of atypical pneumonia and in the therapy of viral diseases as defined above.

In one embodiment in formula (I) R₁, R₂, R₃, R₄, R₅, R₆ is H;
R₃ is —(CH₂)ₙNR₈R₉;
n is 1 to 5, preferably 1 to 3, more preferably 1 to 2,
R₈ is (C₁₋₁₀)alkyl, preferably (C₁₋₂)alkyl,
R₉ is (C₁₋₁₀)alkyl, preferably (C₁₋₂)alkyl;

The salt (S) can be formed, for example, by an acid-base reaction of the base (BA) when carrying out step b) with the acid added to the amine (AM), if an acid addition salt of the amine (AM) is used. The salt (S) may also be added directly in one of steps a), b) and/or c). The direct addition of the salt (S) is preferred if the amine (AM) is not used in the salt form and/or step b) is not carried out.

Preferably, the carbonic acid adduct (KA) remains stable during storage at a temperature of 2 to 10° C. for at least 12 months, more preferably for at least 13 months, even more preferably for at least 20 months, particularly preferably for at least 23 months, and most preferably for at least 27 months.

The carbonic acid adduct (KA) is no longer considered stable if the specific bands of the at least one amine (AM) can be detected by IR spectroscopy, in particular in the solid of the carbonic acid adduct (KA). The specific IR bands of the amine are those bands that are also detected in the IR spectroscopic examination of the pure amine (AM). As long as the amine (AM) is bound in the stable carbonic acid adduct (KA), the specific IR bands of the amine (AM) are not detected.

In individual embodiments, the loss of stability may also be accompanied by an increase in pH, or by the measurement of two melting/decomposition ranges, i.e. a range corresponding to the amine (KA) and a range corresponding to the carbonic acid adduct (KA). The carbonic acid adduct, which has become unstable due to at least partial decomposition into the amine (AM) and CO₂ and/or water, may also experience a change in solution behaviour. The carbonic acid adduct (KA) that has become unstable may prove to be more difficult to dissolve or may be at least partially incompletely dissolved.

The carbonic acid adduct (KA) is preparable by a process comprising the steps a), optionally b), c), d) and e).

In step a), a solution (A) is provided comprising at least one solvent and CO₂ dissolved in the at least one solvent.

The solution (A) comprises at least one solvent and CO₂ in dissolved form. In the context of this invention, CO₂ is understood to mean carbon dioxide. CO₂ in dissolved form is understood in the context of this invention to mean all forms of CO₂ which it enters during dissolution. For example, it is known for aqueous solutions that the dissolved CO₂ can be present in the solution inter alia in equilibrium as CO₂, as carbonic acid, as single or double deprotonated carbonic acid, i.e. as hydrogen carbonate or carbonate.

The solution (A) is obtained by introducing CO₂ into the at least one solvent. The CO₂ can be introduced into the solvent in any suitable form known to a person skilled in the art.

Preferably, gaseous or frozen CO₂ in the form of dry ice, more preferably gaseous CO₂ is introduced into the solvent. Preferably, the gaseous CO₂ or frozen CO₂ is at least food grade, more preferably a grade suitable for pharmaceutical use. The gaseous and/or frozen CO₂ used preferably has a purity of at least 99.5%, even more preferably 99.9%.

Preferably, food quality in the context of this invention means that the relevant regulations of German and European food law are fulfilled. These preferably include Regulation (EC) No 852/2004 and Regulation (EC) No 178/2002 as well as Regulation (EC) No 1333/2008 and Regulation (EU) 231/2012.

Preferably, pharmaceutical quality means that the relevant regulations of the European Pharmacopoeia (Ph. Eur.) are fulfilled.

The CO₂ can also be introduced under pressure, especially when gaseous CO₂ is introduced into the solution. In this context, introduction under pressure means that a pressure greater than atmospheric pressure, preferably greater than 1.01325 bar, is used. For this purpose, the introduction of the CO₂ into the solvent can take place in a container which isolates the solvent from the environment in such a way that a pressure can be generated in the container, in particular via the supply of the CO₂, which is above atmospheric pressure, preferably above 1.01325 bar. The introduction of the CO₂ into the solution, in particular in gaseous form, can take place in one step or at intervals.

A person skilled in the art can use any suitable solvent in step a). Preferably, the solvent used is a polar-protic solvent, more preferably the solvent is water. The solvent may be used in various degrees of purity depending on the intended use of the carbonic acid adduct (KA). For example, water with the purity grade “aqua ad iniectabilia” (water for injection) can be used if the carbonic acid adduct (KA) is to be used for pharmaco-medical purposes.

Step a) may comprise sub-step a1), wherein the solvent is cooled to 3 to 8° C., preferably to 5° C., preferably before the introduction of the CO₂. Cooling may be performed by any method known to a person skilled in the art and identified as suitable. For example, cooling can be done by keeping the solvent in a refrigerator for a sufficiently long time until the solvent is at the target temperature. Likewise, external cooling may be used, for example.

Step a) may comprise sub-step a2), wherein the CO₂ is introduced into the solvent, preferably until a saturation concentration of 3 to 10 g/l is reached, more preferably up to a saturation concentration of 4.5 to 7.5 g/l relative to the total volume of the solution. Preferably, the pH of the solution after saturation with CO₂ is ≤3.0 to ≤6.0, even more preferably ≤4.3 to ≤4.8. Preferably, the CO₂ is dissolved under pressure in sub-step a2), wherein the pressure is 1.5 to 10 bar, more preferably 1.9 to 7 bar, even more preferably 2 to 5 bar.

Step a) may comprise sub-step a3), wherein the solution (A) preferably obtained in sub-step a2) is stored at 1 to 10° C. preferably for at least 30 min, more preferably for at least 50 min, even more preferably for at least 60 min; to at most 5d (120 h). Preferably, the solution (A) preferably obtained in sub-step a2) is stored at 3 to 8° C. for at least 30 min, more preferably for at least 50 min, even more preferably for at least 60 min; up to at most 5d (120 h).

Preferably, step a) comprises all sub-steps a1), a2) and a3).

Preferably, sub-steps a1), a2) and a3) are carried out in the order a2) follows a1) and a3) follows a2).

Optionally, step b) may be carried out wherein the base (BA) other than the amine (AM) is dissolved in the solution (A) to obtain the solution (A1). Preferably, the base (BA) is a hydrogen carbonate or a carbonate, more preferably a hydrogen carbonate, even more preferably sodium hydrogen carbonate.

In step c), the at least one amine (AM) is dissolved in the solution (A) or (A1) to obtain the solution (B).

The at least one amine (AM), as defined above, can be used in step c) both in neutral form and in salt form. Optionally, the at least one amine (AM) may also be used as a mixture of the neutral form of the amine (AM) with the salt form of the amine (AM). Therefore, the at least one amine (AM), may comprise the neutral amine (AM) and/or the salt form of the at least one amine (AM). Preferably, the salt form of the at least one amine (AM) is an acid addition salt, preferably the acid addition salt is a hydrochloride, hydrobromide, hydroiodide, hydrogen sulphate, hydrogen sulphite, hydrogen phosphate, hydromesylate, hydrotosylate, hydroacetate, hydroformate, hydropropanoate, hydromalonate, hydrosuccinate, hydrofumarate, hydroxalate, hydrotartrate, hydrocitrate, hydromaleate, more preferably a hydrochloride or hydrobromide, even more preferably a hydrochloride of the at least one amine (AM).

Preferably, the concentration of the amine (AM) in solution (B) is 0.01 to 0.25 g/ml, preferably 0.03 to 0.20 g/ml, more preferably 0.08 to 0.15 g/ml.

Step c) may comprise sub-step c2), wherein the at least one amine (AM) is dissolved in solution (A), or, if step b) is carried out, in solution (A1) to obtain solution (B).

In one possible embodiment, the ratio of amine (AM) to base (BA), when carrying out step b), in solution (B) is from 2:1 to 5:1, more preferably from 3:1 to 4:1, even more preferably from 3.23:1 to 3.26:1 [g/g]].

In another possible embodiment, the molar ratio of the amine (AM) to the base equivalents of the base (BA), when carrying out step b), in solution (B) is 0.8:1 to 1.5:1, preferably 1.2:1, more preferably 1:1. Base equivalents in this context means that when a monovalent base is used, such as NaHCO₃, the molar ratio of the base (BA) to the amine (AM) corresponds to the ratio given above. When using a divalent base (BA), such as Na₂CO₃, only half the amount of base is required relative to the amount of substance in moles of the base (BA) compared to the use of a monovalent base in order to introduce the same amount of base equivalents. For example, at a ratio of 1:1, if 10 mmol of amine (AM) are used, 10 mmol of NaHCO₃ are required, but only 5 mmol of Na₂CO₃.

In a further embodiment, step b) is carried out and in sub-step c1) the amine (AM) is added in the form of the acid addition salt, wherein the amine (AM) with the acid bound to it is added in an amount such that the acid bound to the amine (AM) is able to neutralise the base (BA) to such an extent that the solution (B) assumes a pH value of 6 to 8.

Step c) may comprise sub-step c2), wherein solution (A) is added to solution (B) to obtain solution (B1).

Preferably, the concentration of the amine (AM) in solution (B1) is 0.01 to 0.25 g/ml, preferably 0.03 to 0.20 g/ml, more preferably 0.08 to 0.15 g/ml.

Step c) may comprise sub-step c3), wherein the solution (B) or, when performing sub-step c2), the solution (B1) is enriched with CO₂. Preferably, the solution (B) is enriched with 2.5 g/l to 9 g/l, more preferably with 5 to 7.5 g/l CO₂.

Step c) may comprise sub-step c4), wherein the solution (B) or, when performing sub-step c2), the solution (B1) is stored at 1 to 10° C., preferably 3 to 8° C., for at least 1 h, preferably 24 h to 120 h, even more preferably 24 to 72 h.

Step c) may comprise sub-step c5), wherein the solution (B) or, when performing sub-step b2), the solution (B1) is enriched with CO₂ preferably to a total concentration of at least 6 g/l, more preferably at least 10 g/l, even more preferably at least 12 g/l, very particularly preferably at least 14 g/l and most preferably at least 15 g/l. Preferably, in sub-step c5), a further 0.4 to 4.7 g/l, more preferably 1 to 3.5 g/l CO₂ is introduced or dissolved into the solution (B) or (B1) until the required total concentration is reached.

The term “total concentration” refers here to the total concentration of dissolved CO₂ in the solution (B) or (B1), including the CO₂ bound in the carbonic acid adduct (KA). The total concentration results additively from the weight increase of the solution by the added CO₂ in all preceding enrichment steps a2) and/or c3), if carried out, and c5), without taking into account CO₂, which is optionally added in the form of hydrogen carbonate or carbonate as base (BA) to the solution.

The enrichment of the solution (B) or the solution (B1) in sub-step c5) with CO₂ to the required total concentration can be carried out at a pressure of 2.5 to 10 bar, preferably of 4 to 10 bar, more preferably of 5 to 10 bar, even more preferably at 6 to 10 bar, most preferably at 6.5 to 10 bar. Preferably, the solution (B) or (B1) has a temperature of 3 to 8° C., more preferably 5° C., when enriched with CO₂ in sub-step c5).

The enrichment of solution (B) or (B1) in sub-steps c3) and c5) can be done in the same way as described for step a).

Preferably, the pH of the solution (B) or, when performing sub-step c2), of the solution (B1) after performing step c5) is ≤7.0.

Preferably, step c) comprises all sub-steps c1), c2), c3), c4) and c5).

Preferably, sub-steps c1), c2), c3), c4) and c5) are carried out in the order c2) follows c1), c3) follows c2), c4) follows c3), c5) follows c4).

In step d), the solution obtained after completing step c) is frozen. Preferably, in step d), the solution B) or, after carrying out sub-step c2), the solution (B1) is frozen.

The solution, preferably solution (B) or (B1), subjected to step d) has a CO₂ content of at least 6 g/l, preferably at least 10 g/l, more preferably at least 12 g/l, even more preferably at least 14 g/l and very particularly preferably at least 15 g/l.

Preferably, the solution obtained after completion of step c), preferably solution (B) or (B1), is frozen at −100° C. to −20° C., more preferably at −90° C. to −30° C., even more preferably at −80 to −40° C. and most preferably at −70 to −50° C.

The freezing of the solution obtained after step c), preferably solution (B) or (B1), can in principle be carried out by any method known to a person skilled in the art and identified as suitable. For example, freezing can be performed by transferring the solution obtained in step c) into a suitable vessel that is immersed in a cooling medium. Preferably, the vessel has a flask shape. Preferably, the vessel containing the solution obtained in step c) is immersed in the cooling medium at an angle of 40°. The cooling medium may, for example, consist of a solvent such as methanol, ethanol or acetone, which is brought to the desired temperature by adding dry ice, or by suitable cooling apparatuses such as cryostats.

Preferably, freezing takes place at atmospheric pressure, more preferably at 1.01325 bar.

Preferably, the solution obtained after completion of step c), preferably solution (B) or (B1), is frozen within 0.3 to 60 minutes, more preferably within 1 to 30 minutes, even more preferably within 1.1 to 10 minutes, most preferably within 1.5 to 5 minutes.

The solution obtained after completion of step c), preferably solution (B) or (B1), is preferably frozen at a cooling rate of 10 to 100 K/min, more preferably at 20 to 80 K/min, even more preferably at 30 to 70 K/min and particularly preferably at 40 to 60 K/min.

Preferably, the vessel in which the solution obtained after completion of step c), preferably solution (B) or (B1), is located during the freezing process is rotated in the cooling medium at 10 to 1000 rpm, more preferably at 50 to 600 rpm, even more preferably at 100 to 400 rpm and particularly preferably at 200 to 300 rpm.

Freezing can be done according to the shell freeze procedure.

In step e), the solution frozen in step d), preferably solution (B) or (B1), is stored at −100 to 0° C. for no longer than 4 days.

Preferably, the solution frozen in step d) is stored in step e), preferably solution (B) or (B1), for 1.5 to 4 days, more preferably for 2.5 to 4 days.

Preferably, the solution frozen in step d) is stored in step e), preferably solution (B) or (B1) at −50 to 0° C., more preferably at −30 to −5° C., even more preferably at −25 to −10° C., particularly preferably at −20 to −15° C.

In principle, storage at the defined temperature can take place in any refrigeration facility known to a person skilled in the art. For example, the storage can be carried out in a freezer or a deep-freeze room.

The process according to which the carbonic acid adduct (KA) can be produced can comprise a further step f), which is carried out after step e). In step f), the solution stored in step e), preferably solution (B) or (B1), is dried to obtain dried carbonic acid adduct (KA).

Preferably, in step), the water is removed from the solution stored in step e), preferably solution (B) or (B1) up to a residual content of <0.8 wt. %, more preferably up to a residual content of <0.1 wt. % relative to the total weight of the dried carbonic acid adduct (KA).

Preferably, in step f), CO₂ not bound in the carbonic acid adduct (KA) is removed from the solution stored in step e), preferably (B) or (B1), up to a residual content of <0.8 wt. %, more preferably up to a residual content of <0.1 wt. % in relation to the total weight of the dried carbonic acid adduct (KA).

Drying can be carried out using all methods known to a person skilled in the art and identified as suitable. Preferably, the drying is carried out by means of freeze-drying, also called lyophilisation. Step d), in the case of using the freeze-drying method, represents the freezing step and step e) the maturing step.

Preferably, the pressure during drying is 0.01 to 30 mbar, preferably 0.02 to 20 mbar, more preferably 0.03 to 10 mbar, even more preferably 0.03 to 0.5 mbar and very particularly preferably 0.05 to 0.1 mbar. Preferably, the pressure is maintained throughout the drying process. Preferably, the pressure defined above is reached during drying within 7 h, more preferably within 5 h and particularly preferably within 4 h from the start of evacuation.

The end point of the drying can be determined by a person skilled in the art from the temperature curve recordings. Preferably, the total drying time in step f) is 10 to 60 h, more preferably 30 to 55 h, particularly preferably 41 to 52 h. The total drying time is defined as the time span between the completion of storage in step e) and the completion of drying in step f).

Preferably, the temperature throughout the drying in step f) is 0 to 20° C., preferably 4 to 18° C., more preferably 8 to 16° C.

Furthermore, the invention comprises a pharmaceutical composition (PZ) comprising the amine (AM), or the carbonic acid adduct (KA) as described above for use in the treatment of atypical pneumonia.

Pharmaceutical compositions also containing the carbonic acid adduct (KA) are described in WO2019/048590.

The pharmaceutical preparation (PZ) in the context of the present invention is basically understood to be a composition comprising the amine (AM) or carbonic acid adduct (KA) and may further comprise other excipients or additives suitable for pharmaco-medical use.

In addition, the pharmaceutical preparation (PZ) may comprise further bases which do not correspond to the amine (AM) and may be different from the base (BA). A person skilled in the art can basically select the additives according to the desired use. In doing so, they will take into account the desired form of application.

The pharmaceutical preparation (PZ) can basically be present in any suitable dosage form. For example, the pharmaceutical preparation (PZ) may be present in capsule form, as a tablet, as a solution, as an ointment, as a cream, as a gel, as a paste, as an enveloping paste or as an active-substance-containing patch.

The pharmaceutical preparation (PZ) can basically be applied in any suitable application form. A person skilled in the art will select a suitable dosage form according to the intended application form. For example, the pharmaceutical preparation (PZ) can be administered orally, by inhalation, by injection, as a patch, cutaneously comprising at least dermal application, by application to the eye, by nasal application, by rectal application and by vaginal application.

When preparing the pharmaceutical preparation (PZ), a person skilled in the art can basically use the methods known in the prior art.

Preferably, the temperature of the mixture of the carbonic acid adduct (KA) and the excipients used and optionally further bases during the preparation of the pharmaceutical preparation (PZ) is less than 60° C., preferably less than 50° C., more preferably 0 to 50° C.

When preparing the pharmaceutical preparation (PZ), preferably in ointment form, dispersing can also be used, preferably by means of an ointment preparer. Here, a speed of <2000 rpm is preferably used.

The carbonic acid adduct (KA) can be triturated into powder, alone or in the presence of other excipients or bases, before processing into the oral dosage forms described below, such as tablets or capsules or semi-solid dosage forms. A person skilled in the art can in principle use the technical means suitable and known for the particular purpose. For example, mortars or similar suitable devices may be used for trituration steps. Preferably, a technical aid is used for the trituration steps that keeps the mechanical stress on the carbonic acid adduct as low as possible. Preferably, the trituration is carried out with a mortar.

The powder obtained in this way can then be pressed into tablets, for example, or filled into commercially available capsules or mixed with suitable excipients and processed into semi-solid dosage forms.

One embodiment of the pharmaceutical preparation (PZ) relates to a pharmaceutical preparation (PZ) which comprises the carbonic acid adduct (KA) and is administered orally. In this embodiment, the pharmaceutical preparation (PZ) is preferably administered in capsules, more preferably in hard gelatin or cellulose capsules, particularly preferably in hard gelatin capsules. Likewise, in this embodiment, the pharmaceutical preparation (PZ) can be applied in tablet form.

Preferably, the pharmaceutical preparation (PZ) in this embodiment comprises at least one excipient (H), preferably selected from starch, in particular corn starch and/or rice starch, dextran, cellulose ester and SiO₂.

Moreover, in this embodiment, the pharmaceutical preparation (PZ) may comprise at least one base (BA1) other than the amine (AM) and identical to or different from the base (BA). Preferably, the base (BA1) is selected from NaHCO₃ or KHCO₃, more preferably NaHCO₃.

Preferably, the general indications given above for the pharmaceutical preparation (PZ) also apply to this embodiment, in particular also for the preparation of the pharmaceutical preparation (PZ) as far as technically applicable for this embodiment.

Preferably, the pharmaceutical preparation (PZ) in this embodiment comprises
a) 1 to 99 wt. %, more preferably 15 to 95 wt. % of the carbonic acid adduct (KA),
b) 0 to 60 wt. %, more preferably 3 to 50 wt. %, of the base (BA1) and 1 to 90 wt. %, more preferably 2 to 75 wt. %, of the excipient (H), in relation to the total weight of the pharmaceutical preparation.

A further embodiment of the pharmaceutical preparation (PZ) relates to a semi-solid pharmaceutical preparation (PZ) which comprises the carbonic acid adduct (KA) and is applied cutaneously. The pharmaceutical preparation (PZ) in this embodiment can be applied, for example, in ointment form, as a cream, as a gel, as a paste, as an enveloping paste or as an active-substance-containing patch.

Preferably, the general information given above for the pharmaceutical preparation (PZ) also applies to this embodiment, in particular also to the production of the pharmaceutical preparation (PZ) as far as technically applicable to this embodiment.

In this embodiment, the pharmaceutical preparation (PZ) preferably comprises at least one excipient (H1) selected from paraffins, in particular thick and thin paraffins, wool wax, wool wax alcohols, hydrophobic base gel, vegetable oils, animal fats, synthetic glycerides, liquid polyalkylsiloxanes, waxes, petrolatum and starch, in particular corn starch, preferably petrolatum.

Viscous paraffins (paraffinum subliquidum) are paraffins that have a viscosity of 110 to 230 mPas, while thin paraffins (paraffinum perliquidum) have a viscosity of 25 to 80 mPas.

Preferably, the pharmaceutical preparation (PZ) in this embodiment comprises:
a) 0.1 to 40 wt. %, preferably 0.4 to 10 wt. % of the carbonic acid adduct (KA) and
b) 60 to 99.9 wt. %, preferably 80 to 96 wt. %, of the excipient (H1) in relation to the total amount of the pharmaceutical preparation (PZ).

A further embodiment of the pharmaceutical preparation (PZ) comprising the carbonic acid adduct (KA) relates to a pharmaceutical preparation which is applied buccally, parenterally, nasally and/or by inhalation.

In a buccally applied embodiment, the carbonic acid adduct (KA) is administered in the form of a preparation comprising the carbonic acid adduct (KA) and polyvinylpyrrolidone. Preferably, the mass ratio of carbonic acid adduct (KA) to polyvinylpyrrolidone is 1:1 to 1:10, more preferably 1:3. Polyvinylpyrrolidones of different chain length may be used. Preferably, the preparation contains water as solvent. More preferably, the water contains CO₂. Even more preferably, the water contains at least 3 g/l CO₂, more preferably 3-7 g/l CO₂, most preferably 6-7 g/l CO₂.

Preferably, the general information given above for the pharmaceutical preparation (PZ) also applies to this embodiment, in particular also to the preparation of the pharmaceutical preparation (PZ) as far as technically applicable to this embodiment.

Preferably, the pharmaceutical preparation (PZ) in this embodiment is present as a solution (A2) comprising the carbonic acid adduct (KA), dissolved CO₂ and at least one excipient (H2).

The excipient (H2) is preferably selected from an alkali halide or alkaline earth halide, more preferably NaCl and MgCl₂, even more preferably NaCl. The excipient (H2) may be identical to the salt (S). Quantities relating to the excipient (H2), insofar as this is identical to the salt (S) in individual embodiments, refer in the context of this invention to additional quantities of the excipient (H2) which have not been introduced into the pharmaceutical preparation (PZ) in the form of the salt (S) as part of the carbonic acid adduct (KA).

Preferably, the solution (A2) is obtained by introducing CO₂ into a solvent. Preferably, the solvent is water. Preferably, the CO₂ is introduced into the solvent at a temperature of 0 to 8° C., more preferably at 0 to 5° C., to produce the solution (A2). The CO₂ can be introduced into the solvent in the form of the gas as well as in solid form, for example as dry ice. Preferably, the CO₂ is introduced into the solvent in the form of the gas. The CO₂ can also be introduced into the solution under pressure until the desired concentration is reached, as described above for sub-step a2).

Preferably, the CO₂ is introduced into the solvent to a concentration of at least 3 g/l, more preferably up to 4 g/l, even more preferably 4 g/l to 8 g/l, to prepare the solution (A2).

Preferably, the CO₂ used to prepare the solution (A2) has at least a purity of 99.9%, more preferably also a quality suitable for pharmaceutical applications as defined above.

Preferably, the pharmaceutical preparation comprises (PZ) in this embodiment, insofar as it is obtained by dissolving the carbonic acid adduct (KA) in the solution (A2),
a) 0.05 to 100 mg/ml, more preferably 0.08 to 50 mg/ml of the carbonic acid adduct (KA) and
b) 0 to 20 mg/ml, more preferably 3 to 10 mg/ml of excipient (H2) in each case in relation to the total volume of the pharmaceutical preparation (PZ).

### EXAMPLES OF THE INVENTION

### 1. Practical Example 1, Preparation of the Carbonic Acid Adduct (KA)

### 1.1 Materials

Amine (AM): 68.8 to 110.1 g procaine hydrochloride (e.g. ultrapure, for use as an active pharmaceutical substance; Ph. Eur. or of a quality suitable for this purpose).
Base (BA): 22.2 to 33.9 g sodium hydrogen carbonate (e.g. ultrapure or of a quality suitable for this purpose),
Solvent: 630 to 900 ml water (aqua ad iniectabilia).
CO₂: min. 12.0 g/l carbon dioxide from pressurised gas steel cylinders (CO₂ in suitable quality)
Dry ice for the preparation of refrigeration mixtures and for cooling
Methanol, techn. for the preparation of refrigeration mixtures

### 1.2 Step a)

Water (e.g. aqua ad iniectabilia) is poured into a cleaned plastic pressure bottle up to the mark (approximately 800 to 900 ml) and precooled to 5° C. for at least 1 h in the refrigerator (3 to 8° C.) or by means of external cooling.

A carbon-dioxide-saturated carbonic acid solution is prepared. To do this, CO₂ is introduced at intervals under pressure (1.6 to 8 bar) into the precooled water. The hissing sound (escaping gas via the pressure relief valve) indicates saturation of the solution with CO₂. Saturation is controlled by weight until 4.0 to 6.0 g CO₂ (corresponding to 4.5 to 7.5 g/l) are dissolved. The saturated solution has a pH value of ≤4.3 to 4.8. This carbonated water is immediately sealed and stored in the refrigerator for at least 1 h.

### 1.3 Step b)

In a second plastic pressure bottle, 21.2 g of sodium hydrogen carbonate is placed, mixed with 320 ml of cooled water containing CO₂ and dissolved while swirling.

### 1.4 Step c)

To this solution is added the equivalent amount of solid procaine hydrochloride at a constant temperature, forming an almost neutral solution which, after the addition of a further 320 ml of cold carbonated water, gives a clear weakly acidic solution. The solution is enriched with CO₂. The solution thus prepared is stored in the refrigerator for at least 1 h.

The solution is then conditioned again with CO₂ until a CO₂ concentration of 12 g/l is reached in the solution The pH value is checked using pH indicator strips. The pH value is ≤6.6.

### 1.5 Step d)

Round-bottom flasks are pre-cooled. For freezing, the reaction solution is measured in a pre-cooled measuring cylinder, transferred in portions to round-bottom flasks and frozen by immersion in a dry ice/methanol refrigeration mixture (<−60° C.) according to the shell-freezing method within 1.5-3.5 min per flask (˜200 rpm). The immersion angle of the flask on the rotary evaporator is set to approximately 40°.

### 1.6 Step e)

The flasks with the frozen material are sealed with a ground-glass stopper and temporarily stored in a freezer at −15 to −20° C. for 2 to 4 days.

### 1.7 Step f)

The temperature-controlled flasks are encased in polystyrene containers, which are pre-cooled and immediately connected individually to an evacuated (0.060±0.01 mbar, approximately −46° C., leak test) freeze-drying unit, via a flexible rubber cone. The valve cocks are carefully opened and the individual pistons are placed under vacuum. Lastly, all pistons must be evacuated.

To monitor the process, temperature sensors are placed at the bottom of the polystyrene jacket to record the entire temperature curve throughout the drying process. Before the start of lyophilisation, the temperature sensors indicate temperatures of <−5° C.

During lyophilisation, the pressure is 0.07±0.02 mbar. This sublimation pressure is reached within 4 h and maintained during the entire lyophilisation time. The temperature of the cooling chamber is kept at 9 to 15° C. during the entire drying process. The end point of the lyophilisation is determined graphically from the temperature curve recordings. The total drying time was a maximum of 52 h. The dry lyophilisate is transferred to an amber glass jar with a twist-off lid, provided with a desiccant bag and stored in the refrigerator at 0 to 15° C.

### 2. Examples of Pharmaceutical Preparations (PZ)

### 2.1 Capsules and Tablets

The following is an exemplary description of the composition of the pharmaceutical preparation (PZ) according to the invention in the embodiment as a capsule or tablet for procaine as amine (AM). For the production of the capsules, commercially available plug capsules in the commercially available sizes (5 to 000) can be used, which are filled with the powder containing carbonic acid adduct (KA), comprising procaine as amine (AM) (trituration of the active substance carbonic acid adduct (KA), comprising procaine as amine (AM), optionally with additives, fillers and flow regulators). The carbonic acid adduct (KA) was prepared according to Example 1. It has been shown that hard gelatine capsules are more suitable than cellulose capsules with regard to stability. For example, the filled hard gelatine capsules, exemplified by hard gelatine capsules with 60 and 100 mg active substance according to Table 1, did not show any changes even after 12 months of storage in the refrigerator and are thus stable (FIG. 2). The stability was examined by means of IR spectroscopy. In the case of the hard gelatine capsules, no procaine was detected by IR spectroscopy within the 12-month period, whereas a procaine band was measured in the IR spectrum of the cellulose capsules after only a few days. The content was determined at room temperature with UVVIS spectroscopy. According to Pharmacopoeia Europaea point 2.9.6, a tolerance range of ±15% is prescribed for prescription medicinal products in relation to the total content including by-products.

The valid and generally accepted pharmaceutical rules for the production of (prescription) medicinal products are applied (e.g. Pharmacopoeia Europaea, German Pharmaceutical Codex).

TABLE 1
Example composition of capsules with NaHCO₃ added and carbonic
acid adduct (KA) as active substance produced according
to practical example 1.

| | | | | |
|---|---|---|---|---|
| Active substance amount [mg] | 30 | 60 | 100 | 120 |
| Addition, for example NaHCO₃ | 21 | 42 | 71 | 84 |
| Filler, for example corn starch incl. SiO₂ | 120 | 90 | 120 | 90 |
| Capsule size | 1 | 1 | 0 | 0 |

TABLE 2
Example composition tablet with NaHCO₃ added and carbonic acid adduct
(KA) as active substance prepared according to practical example 1.

| | | | | |
|---|---|---|---|---|
| Active substance amount [mg] | 30 | 60 | 100 | 120 |
| Addition, for example NaHCO₃ | 21 | 42 | 71 | 84 |
| Filler, for example corn starch incl. SiO₂ | 2.5 | 5 | 8.5 | 10 |

### 2.2 Ointments

In the following, the composition of the pharmaceutical preparation (PZ) according to the invention in the ointment embodiment is explained by way of example for procaine as amine (AM) in the carbonic acid adduct (KA). In the preparation of the ointment, the applicable and generally accepted pharmaceutical rules for the preparation of (formulation) medicinal products are applied (e.g. Pharmacopoeia Europaea, Deutscher Azneimittel-Codex). Major shear forces are avoided during the preparation of the ointment. In addition, the temperature is also kept below 60° C. locally during preparation. Thus, the mortared carbonic acid adduct (KA) comprising procaine as amine (AM) is introduced into the ointment base, e.g. Vaseline, in a mortar or a melamine bowl, which are temperature-controlled by means of a water bath to a temperature of 40 to 45° C. Alternatively, it is also possible to use electric mixing systems, such as those used in normal pharmacy operations.

TABLE 3
Example composition ointment and carbonic acid adduct (KA) as
active substance prepared according to practical example 1.

| | | | | | |
|---|---|---|---|---|---|
| Content [%] | 0.5 | 1.0 | 1.25 | 2 | 4 |
| Active substance amount [mg] | 25 | 50 | 62.5 | 100 | 200 |
| Ointment base [g] (for example Vaseline) | 5 | 5 | 5 | 5 | 5 |

### 2.3 Parenteral Solutions

For the preparation of a parenteral solution containing the carbonic acid adduct (KA) comprising procaine as amine (AM), the required quantity of water (aqua ad iniectabilia) is cooled to approximately 5±3° C. in a suitable vessel with a magnetic stir bar or similar and left at this temperature. The water is enriched with gaseous carbon dioxide of the required quality to approximately 3.2 g/l. In this CO₂-containing water, the appropriate amount of carbonic acid adduct (KA) comprising procaine as amine (AM) and sodium chloride is dissolved for an isotonic content.

Alternatively, water temperature-controlled to approximately 5±3° C. is enriched under pressure in a closed system with CO₂ in such a way that a clear excess is present (4.5 to 7.5 g/l). The corresponding amounts of carbonic acid adduct (KA) comprising procaine as amine (AM) and sodium chloride are also added to this carbonated water.

This cold solution, provided with the carbonic acid adduct (KA) comprising procaine as amine (AM) and sodium chloride, is sterile-filtered under suitable spatial conditions and filled into appropriate vials. The applicable and generally accepted pharmaceutical rules for the preparation of (formulation) medicinal products are applied (for example Pharmacopoeia Europaea).

TABLE 4
Example compositions for parenteral preparations with
NaCl addition and carbonic acid adduct (KA) as active
substance prepared according to practical example 1.

| | Infusion | | | Injection | | |
|---|---|---|---|---|---|---|
| Content [%] | 0.1 | 0.2 | 0.3 | 1 | 2 | 3 |
| Active substance amount [mg] | 50 | 100 | 150 | 50 | 100 | 150 |
| Addition (NaCl) [mg] | 440 | 430 | 425 | 37 | 29 | 25 |
| Total volume [ml] | 50 | 50 | 50 | 5 | 5 | 5 |

### 2.4 Buccal Application as Pump Spray

Dissolve 285 mg ProcCluster (carbonic acid adduct according to example 1) and 852 mg PVP25 (mass ratio 1:3) in 5 ml CO₂ water (e.g. commercial mineral water, CO₂₋) and apply using a pump spray bottle. PVP=polyvinylpyrrolidone, also called polyvidone or povidone. Other PVP products can be used, for example PVP30 and PVP17→other ratios (expedient from 1:1 to 1:10 in relation to amount of ProcCluster, applies to all PVP used)

PVP: CAS No. Designation 9003-39-8 polyvinylpyrrolidone (2-pyrrolidinone, 1-ethenyl-, homopolymer) EINECS No.: 618-363-4-REACH Reg. No.:—(Polymer)—INCI Name: PVP.

### 3. Solubility in Octanol

Solubility tests were carried out in octanol and the content in the organic phase was investigated by UV/VIS spectroscopy and the pH values.

TABLE 5
Solubility of different dosage forms

| Substance | Content | pH value |
|---|---|---|
| Procaine | completely dissolved (100%) | 8.5 |
| Procaine hydrochloride | 8% | 4.5 |
| Carbonic acid adduct of | 74% | 7.5 |
| procaine | | |

The pH values determined prove that the carbonic acid adduct of procaine is soluble in the organic phase and thus membrane-permeable as such and not converted into procaine. Likewise, the UV/VIS spectroscopic content measurement shows that the carbonic acid adduct of procaine is more similar to procaine than to ProcHCl in terms of lipophilicity. It thus behaves like the basic component—the lipid-soluble base that is formed depending on the pH value. The carbonic acid adduct of procaine exhibits this property independently of the pH, i.e. it does not have to be converted into the lipophilic form by a pH change as is the case with ProcHCl. ProcHCl has a significantly lower value, which at 8% is in the order of magnitude for protein binding of 6%.

### 4. Test for Antiviral Activity

### 4.1 Test for Antiviral Activity Against Influenza Viruses

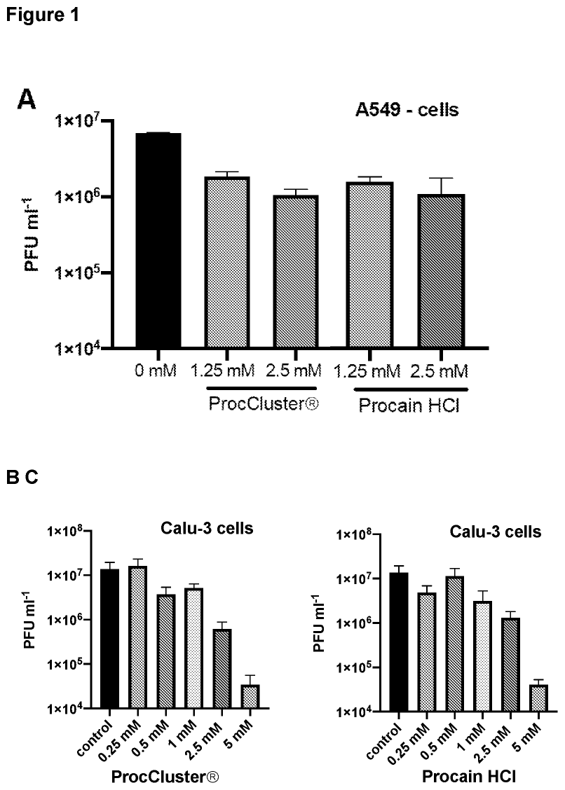

A549 cells were pre-treated for 30 min with the indicated concentrations of ProcCluster® (=carbonic acid adduct (KA) according to Example 1), or procaine HCl dissolved in DMEM_{INF} (0.2% BSA, 1 mM MgCl₂, and 0.9 CaCl₂), pre-treated and then infected for 30 min with influenza A/Puerto Rico/8/34 (MOI 0.1) in PBS_{INF} (0.2% BSA, 1 mM MgCl₂, 0.9 CaCl₂, 100 U ml⁻¹ penicillin and 0.1 mg ml⁻¹ streptomycin). The cells were then washed and incubated in culture medium including inhibitor until 24 h after infection. Virus titres in the supernatant were determined by standard plaque assay on MDCK cells. FIG. 1 (A) shows two independent experiments with biological duplicates.

The virus titres were reduced by up to one log level, i.e. up to 90%, by addition of the carbonic acid adduct (KA) from Example 1 compared to untreated samples.

### 4.2 Test for Antiviral Activity Against SARS-CoV-2

Calu-3 cells were pre-treated for 30 min with the indicated concentrations of FIG. 1 (B) ProcCluster® (=carbonic acid adduct (KA) according to Example 1), or FIG. 1 (C) procaine HCl dissolved in DMEM (10% FCS) and then infected with SARS-CoV-2 (MOI 0.5) in DMEM (10% FCS) including the corresponding inhibitor for 2 h. The cells were washed and incubated for 24 h after infection in DMEM (10% FCS) including the corresponding inhibitor. Cells were then washed and incubated in DMEM (10% FCS) including the indicated concentration of the corresponding inhibitor until 24 h post-infection. Virus titres in the supernatant were determined by plaque assay on Vero-76 cells. An experiment with biological duplicates is shown.

The virus titres were reduced by up to one log level, i.e. up to 90%, by addition of the carbonic acid adduct (KA) from Example 1 compared to samples without addition of the carbonic acid adduct (KA) from Example 1.

### LITERATURE

[1] E. Mutschler. G. Geisslinger, H. K. Kroemer, S. Menzel, P. Ruth; “Mutschler Arzneimittel-wirkungen, Pharmakologie, Pharmakologische Wirkungen Toxikologie”, Wissenschaftlicher Verlagsgesellschaft, 10th edition, 2013
[2] https://′flexikon.doccheck.com/de/Acute_respiratory_distress_syndrome, aufgerufen am 23. März 2020
[3] Pecher S., Böttiger B. W., Graf B., Hollmann M. W.; Anaesthesist 2004; 53; 316-325

1.-15. (canceled)

16. A method of treating atypical pneumonia in a subject in need thereof, the method comprising the step of:
administering to the subject a therapeutic amount of an amine of General Formula (I) or a salt thereof, 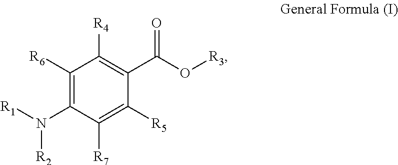
wherein in General Formula (I)
R₁ is H, (C₁₋₁₀)alkyl, (C₂₋₁₀)alkenyl, (C₅-C₁₄)aryl, or (C₅-C₁₀)heteroaryl;
R₂ is H, (C₁₋₁₀)alkyl, (C₂₋₁₀)alkenyl, (C₅-C₁₄)aryl, or (C₅-C₁₀)heteroaryl;
R₃ is —(CH₂)ₙNR₈R₉,
wherein n is an integer 1, 2, 3, 4, or 5;
R₈ is a (C₁₋₁₀)alkyl;
R₉ is a (C₁₋₁₀)alkyl;
R₄ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₅ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₆ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl; and
R₇ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl.

17. A method of treating atypical pneumonia in a subject in need thereof, the method comprising the step of:
administering to the subject a therapeutic amount of a carbonic acid adduct represented by at least one of General Formulae (II), (III), and (IV), 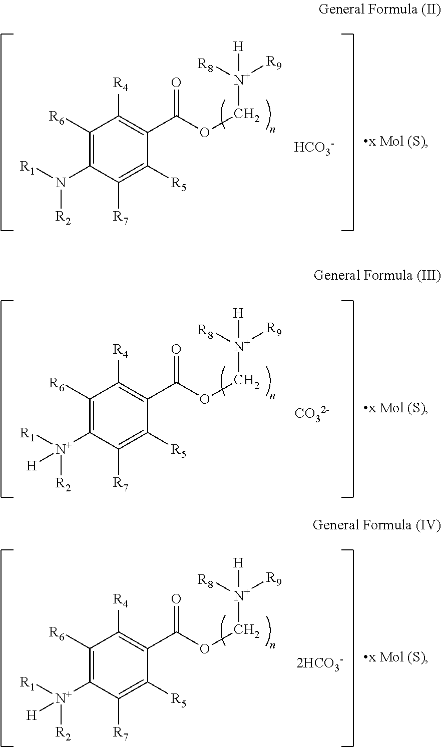
wherein in General Formulae (II), (III), and (IV),
R₁ is selected from the group consisting of H, (C₁₋₁₀)alkyl, (C₂₋₁₀)alkenyl, (C₅-C₁₄)aryl, and (C₅-C₁₀)heteroaryl;
R₂ is selected from the group consisting of H, (C₁₋₁₀)alkyl, (C₂₋₁₀)alkenyl, (C₅-C₁₄)aryl, and (C₅-C₁₀)heteroaryl;
R₃ is —(CH₂)ₙNR₈R₉,
wherein n is an integer 1, 2, 3, 4, or 5,
R₈ is a (C₁₋₁₀)alkyl,
R₉ is a (C₁₋₁₀)alkyl;
R₄ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₅ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₆ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₇ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
x is 0.5 to 30; and
(S) is a salt.

18. A method of treating atypical pneumonia in a subject in need thereof, the method comprising the step of:
administering to the subject a therapeutic amount of an amine-carbonic acid adduct;
wherein the amine-carbonic acid adduct solution is made by a method comprising the steps of:
providing a solution comprising at least one solvent and CO₂;
adding an amine to the solution to form an amine-carbonic acid adduct solution;
wherein the amine is represented by the General Formula (I) or a salt thereof, 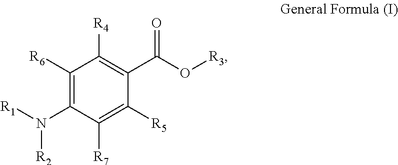
wherein in General Formula (I)
R₁ is H, (C₁₋₁₀)alkyl, (C₂₋₁₀)alkenyl, (C₅-C₁₄)aryl, or (C₅-C₁₀)heteroaryl;
R₂ is H, (C₁₋₁₀)alkyl, (C₂₋₁₀)alkenyl, (C₅-C₁₄)aryl, or (C₅-C₁₀)heteroaryl;
R₃ is —(CH₂)ₙNR₈R₉,
wherein n is an integer 1, 2, 3, 4, or 5;
R₈ is a (C₁₋₁₀)alkyl;
R₉ is a (C₁₋₁₀)alkyl;
R₄ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₅ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
R₆ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl; and
R₇ is H, halogen, (C₁₋₁₀)alkyl, (C₂₋₁₀)alkenyl, (C₅-C₁₄)aryl, (C₅-C₁₀)heteroaryl, or —O(C₁₋₁₀)alkyl;
lyophilizing the solution; and
storing the lyophilizate at a temperature of at most 15° C.

19. The method of claim 18, further comprising the step of dissolving a base in the solution comprising at least one solvent and CO₂.

20. The method of claim 18, wherein the amine represented by the General Formula (1) is selected from the group consisting of 4-aminobenzoic acid-2-(N,N-diethylamino)ethyl ester (procaine), 4-aminobenzoic acid ethyl ester (benzocaine), 2-(diethylamino)ethyl 4-amino-2-chlorobenzoate (chloroprocaine), 4-amino-3-butoxybenzoic acid-2-diethylaminoethyl ester (oxybuprocaine), and (2-(dimethylamino)ethyl)-4-(butylamino)benzoate (tetracaine).

21. The method of claim 18, wherein the amine represented by the General Formula (1) is selected from the group consisting of 4-aminobenzoic acid-2-(N,N-diethylamino)ethyl ester (procaine), 4-aminobenzoic acid ethyl ester (benzocaine), 2-(diethylamino)ethyl 4-amino-2-chlorobenzoate (chloroprocaine), 4-amino-3-butoxybenzoic acid-2-diethylaminoethyl ester (oxybuprocaine), and (2-(dimethylamino)ethyl)-4-(butylamino)benzoate (tetracaine); and
wherein the salt (S) is a salt composed of at least one cation selected from Na⁺, K⁺, Li⁺, Mg²⁺, Zn²⁺, Fe²⁺, Fe³⁺, and Mn²⁺, and at least one anion selected from Cl⁻, Br⁻, J⁻, F⁻, SO₄²⁻, SO₃²⁻, HSO₄_{^{−}}HSO₃_{^{−}}HCO₃_{^{−}}, CO₃²⁻, PO₄³⁻, HPO₄²⁻, H₂PO₄, SiO₄⁴⁻, AlO₂_{^{−}}, SiO₃_{^{−}}, and [AlO₂)₁₂(SiO₂)₂]².

22. The method of claim 18, wherein the step of providing a solution comprising at least one solvent and CO₂ further comprises the step of cooling the at least one solvent to a temperature of 3 to 8° C.

23. The method of claim 18, wherein the step of providing a solution comprising at least one solvent and CO₂ further comprises the step of dissolving CO₂ into the at least one solvent at a saturation concentration of 3-10 g/L.

24. The method of claim 18, wherein in the step of providing a solution comprising at least one solvent and CO₂, the pH of the solution is less than 6.0.

25. The method of claim 18, wherein the step of providing a solution comprising at least one solvent and CO₂ further comprises storing the solution at a temperature of 1 to 10° C. for 30 minutes to 5 days.

26. The method of claim 19, wherein the base is selected from the group consisting of hydrogen carbonate, sodium carbonate, and sodium hydrogen carbonate.

27. The method of claim 18, wherein in the step of adding an amine to the solution to form an amine-carbonic acid adduct solution, the concentration of CO₂ in the solution is at least 15 g/L.

28. The method of claim 18, wherein the step of adding an amine to the solution to form an amine-carbonic acid adduct solution further comprises the step of adding the solution comprising at least one solvent and CO₂ to the amine-carbonic acid adduct solution.

29. The method of claim 18, wherein the step of adding an amine to the solution to form an amine-carbonic acid adduct solution further comprises the step of enriching the amine-carbonic acid adduct solution with CO₂ to a concentration of at least 15 g/L.

30. The method of claim 18, wherein the step of adding an amine to the solution to form an amine-carbonic acid adduct solution further comprises the step of storing the amine-carbonic acid adduct at 1 to 10° C. for at least one hour.

31. The method of claim 18, wherein in the step of adding an amine to the solution to form an amine-carbonic acid adduct solution, the concentration of the amine is 0.01 to 0.25 g/mL.

32. The method of claim 29, wherein the pH of the solution after enriching with CO₂ is less than 7.

33. The method of claim 26, wherein in the step of adding an amine to the solution to form an amine-carbonic acid adduct solution, the ratio of the amine to the base is about 2 to 5.

34. The method of claim 18, wherein in the step of adding an amine to the solution to form an amine-carbonic acid adduct solution, the amine is selected from the group consisting of hydrohalide, hydrogen sulphate, hydrogen sulphite, hydrogen phosphate, hydromesylate, hydrotosylate, hydroacetate, hydroformate, hydropropanoate, hydromalonate, hydrosuccinate, hydrofumarate, hydroxalate, hydrotartrate, hydrocitrate, and hydromaleate.

35. The method of claim 18, wherein the method of making the amine-carbonic acid adduct solution further comprises the step of removing the solvent of the stored solution to obtain dried amine-carbonic acid adduct.

36. The method of claim 35, wherein the solvent has a residual content of less than 0.8 wt % of the total weight of the dried amine-carbonic acid adduct.

37. The method of claim 18, wherein the CO₂ not bound to the amine-carbonic acid adduct is removed from the solution to a residual content of less than 0.8 wt % of the total weight of the dried product.

38. The method of claim 18, wherein in the step where the solution is lyophilized,
the pressure is 0.01 to 30 mbar,
the pressure is reached within 10 hours from the start of evacuation,
the temperature throughout drying is 0 to 20° C., and
the total drying time is 10 to 60 hours.

39. The method of claim 18, wherein the atypical pneumonia is selected from the group consisting of:
a bacterial atypical pneumonia, optionally caused by a bacterium selected from the group comprising mycoplasma, legionella, and chlamydia;
a viral atypical pneumonia, optionally caused by a virus selected from the group comprising coronaviruses, SARS CoV-2, influenza viruses, adenoviruses, and respiratory syncytial viruses; and
an atypical pneumonia caused by fungi, optionally caused by a fungus selected from the group comprising Aspergillus, Pneumocystis and Candida.

40. The method of claim 18, wherein the method is used for the therapy of viral diseases caused by a virus selected from the group comprising RNA viruses, coronaviruses, SARS CoV-2, influenza viruses, adenoviruses, and respiratory syncytial viruses.

41. The method of claim 18, wherein the subject does not require intensive medical treatment.

42. The method of claim 18, wherein administration is performed orally, buccally, parenterally, nasally, by inhalation, or cutaneously.
